(19) United States
(12) Patent Application Publication
(10) Pub. No.: US 2009/0123548 A1
(43) Pub. Date: May 14, 2009

(54) Device for administering aromatherapy

(76) Inventor: Susan Reimer Tom, New York, NY (US)

Correspondence Address: Susan Reimer Tom, 310 West 85th Street, New York, NY, 10024, US

(21) Appl. No.: 11/975,891

(22) Filed: Oct. 22, 2007

(51) Int. Cl. A61K 36/752; A61K 36/53; A61K 36/537; A61K 36/534; A61K 36/328; A61K 36/899; A61K 36/906; A61K 36/28; A61K 36/48; A61K 36/73; A61K 36/13

(52) U.S. Cl. 424/489; 424/736; 424/745; 424/764; 424/746; 424/770; 424/765; 424/750; 424/748; 424/756; 424/725; 424/747; 424/757

(57) ABSTRACT

A device for administering aromatherapy to a subject and the method of using the device are disclosed. The device comprises an aromatherapy composition housed within a container. The container has at least one opening or aperture through which the subject may breathe in or inhale the aromatic and volatile components of the aromatherapy composition through the mouth and/or nose. The aromatherapy composition comprises a particulate component comprising one or more irregularly- or regularly-shaped particles, granules, beads or the like. In addition, the aromatherapy composition has a volatile component comprising an amount of an essential oil-containing composition. The volatile component is that portion of the essential oil-containing composition that is retained by the particulate component after contacting the particulate component with the essential oil-containing composition and removal of the portion that is not retained by the particulate component.

The present invention is directed to a device for administering aromatherapy. The device is in a form suitable for a subject to inhale or breathe in the aromatic and volatile components of an aromatherapy composition comprising scented oils, and more particularly scented essential-oils. The aromatherapy composition is housed or contained in any type container, vessel, or vial that can be sealed with a cap, or a type of orifice reducer/closer, and, optionally, an additional cap. The inhaler disclosed and claimed herein may be used for the inhalation of essential oils. Inhalation is an accepted and approved delivery method in the practice of aromatherapy—the use of essential oils for health benefits to mind and body.

This is the first time that an inhaler encompassed by this description has been assembled in this particular way to achieve the benefits of aromatherapy. In addition, persons of ordinary skill in the arts related to preparing, packaging and commercializing a variety of fragrance-related products would appreciate that the device disclosed and claimed herein may be modified or adapted for additional uses based on the release of aromatic and volatile components of essential oils from the device. For example, air freshening of a room, closet, drawer, hamper, and the like.

### BACKGROUND OF INVENTION

Essential oils are present in tiny droplets between the cells of certain plants, roots and flowers. They may be prepared, i.e., isolated or extracted, from plants by various methods, dependent on choice of the source plant tissue. The most common method is the use of distillation with water, steam or a combination thereof to prepare the essential oil-containing botanical extracts. In an alternative method for producing citrus-based oils, the skins of orange, lemon, lime, etc are cold pressed to crush the oil glands prior to extraction. In still another alternative directed toward the processing of flower petals, solvent extraction is a key feature of the procedure. Thus, depending on the specific plant to be extracted and specific tissue of that plant, various methods are employed by persons of ordinary skill in the art.

The essential oil obtained by these methods is a highly concentrated liquid that is aromatic, potent and volatile. The oil often bears the name of the plant from which it is derived; for example, rose oil or peppermint oil. Such oils were referred to as “essential” because it was believed that they represent the very essence of aroma and flavor. One of the quickest and most effective ways of experiencing the benefits of essential oils is through inhalation.

There have been few rigorous clinical studies systematically examining the effectiveness of aromatherapy. However, aromatherapy is becoming increasingly popular as a complementary therapy for transiently reducing anxiety (Cooke, B., and Ernst, E., (2000) British Journal of General Practice 50: 493-496). Another study suggests that aromatherapy may have broader psychotherapeutic value (Perry, N., and Perry, E., (2006) CNS Drugs, 20:257-280).

Other studies focusing on physiological processes also indicate potential therapeutic effects of aromatherapy. For example, one study indicates that aromatherapy may improve the immune function of cancer patients (Kuriyama, J., et al., (2005) Evidence-based Complementary and Alternative Medicine 2: 179-184).

Additional studies focusing on specific cell-types and pathogens have also been recently reported. In an inflammation mouse-model, it was shown that geranium seed oil suppressed experimentally-induced neutrophil accumulation thereby inhibiting the induced acute and chronic inflammatory responses (Maruyama, N., et al., (2005) J. Inflammation 2:1-7). Further, various essential oils have demonstrated anti-bacterial effects (Prabuseenivasan, S., et al., BMC Complementary and Alternative Medicine, 6:39-46) as well as anti-viral effects toward acyclovir-resistant Herpes Simplex type 1 (Schnitzler, P., (2007) Antimicrobial Agents and Chemotherapy, 51:1859-1862).

The practice of aromatherapy involves, inter alia, instructing a human subject to perform conscious deep-breathing exercises or regimens while inhaling essential oils. This is believed to place a person in a condition to anchor and recall (or trigger) desired states such as relaxation, confidence, focus, joy, serenity. In order to achieve this in a practical manner while being able to work with a number of subjects during a period of time, an easily portable, reliable, light-weight, no spill, and long-lasting vehicle for individual inhalation of essential oils was needed. This inhaler described herein is a response to that need. It is further a benefit of the inhaling device that the aromatherapy composition not contain levels of harmful chemicals that may be inhaled during deep-breathing regimens.

### SUMMARY OF THE INVENTION

An object of the invention is to provide an inhaler—a device that allows a subject or user to inhale the scent of essential oils at their own chosen rate, and according to their own needs. This would be understood by persons of ordinary skill in the art to be distinct from current practice of breathing in the aromatic oils as they are diffused into the general environment in a nonspecific manner that cannot be regulated by each individual. Such would be the case when the essential oils are delivered in a general, non-targeted manner with a room diffuser, nebulizer, vaporizer or air system filter, rather than specifically targeting a single individual who can initiate or terminate the inhalation of the aromatic oils at will.

In one embodiment, the device of the invention encompasses a container that can be held in a person's hand. The container comprises an amount of an aromatherapy composition comprising a particulate component and a volatile component. The particulate component is at least one particle which is capable of absorbing, binding, or in some other manner, retaining an amount of a composition comprising at least one essential oil. This retained amount of the essential oil-containing composition is the volatile component of the aromatherapy composition. Further, the interactions between the particulate material and the volatile or aromatic substances are not irreversible. Thus, under suitable conditions an amount of the volatile or aromatic substances may be released to the ambient atmosphere. Alternatively, the volatile or aromatic substances may be inhaled by a subject either via the nose or mouth.

The material released may be conventional fragrance or suitably prepared aromatic substances such as essential oils. The container further comprises one or more openings from which the aromatic and volatile components of the essential oils can leave the container and are inhaled by a user.

In another embodiment, the particles are contacted with a composition comprising one or more essential oils under conditions wherein the particles retain a portion of the composition. The amount retained is preferably sufficient so that at the desired time, the combined particle-essential oil composition (i.e., the aromatherapy composition) may release an effective amount of the volatile or aromatic substances from the oil to either the atmosphere or directly to a subject via inhalation.

In another embodiment, the particulate component is composed of substantially rounded, bead-like particles. These bead-like particles may be porous so as to absorb or otherwise retain an aromatic substance within the bead-like particle's interior. Suitable materials include, but are not limited to natural material such as wood, clay, or particles of absorbent pebble-shaped particles of minerals.

A further embodiment of the inventive subject matter disclosed herein encompasses an essential-oil retaining particle. Expressed another way, the inventive subject matter is directed to an aromatherapy combination comprising (1) a particulate component, and (2) a volatile component, i.e., one or more essential oils retained by the particle.

Additional embodiments may substitute the natural material particles with those made of synthetic polymers. Thus, in a generic sense, the beads may be prepared from plastics or other suitable polymers. In other embodiments, the particulate bead-like particles may be prepared using a mixture of natural and synthetic materials. Persons of ordinary skill in the art would recognize that functional particles may be made of plastics, ceramics, clays and similar porous materials.

Further embodiments of the particulate materials include particles that are formed from compressing granulated or powdered materials. The powders, in turn, could be composed of grindings of natural or synthetic materials.

### BRIEF DESCRIPTION OF DRAWINGS

FIG. 1 is a non-limiting illustration of an embodiment of a device for administering aromatherapy. Several features are labeled to illustrate their basic functional and spatial inter-relationships as described. The curved dashed lines indicate the volatile component being released through the container opening. Note that an orifice reducer, e.g., a cap, is not shown in the closed position whereby it would seal off the opening or aperture. The features of the device may be modified in various ways and still be encompassed by the invention.

### DETAILED DESCRIPTION

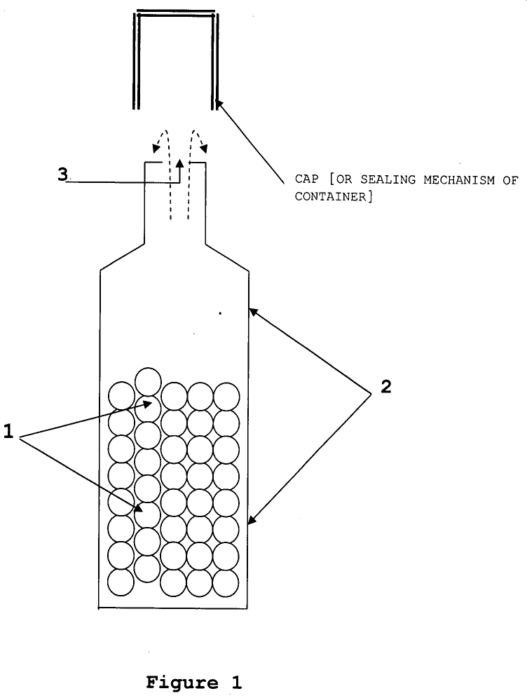

FIG. 1 provides an illustrative view of an embodiment of the aromatherapy device described and claimed herein. The device comprises a container or vessel (2) having an internal space in which is an aromatherapy composition (1). The aromatherapy composition comprises two functionally distinct components (not indicated in FIG. 1). These are (a) a particulate component, and (b) a volatile component. The particulate component comprises at least one particle that has been contacted with at least one essential oil. The at least one essential oil comprises an aromatic component that may be released to the ambient atmosphere from where it may be inhaled by an individual or subject. The component that is released and inhaled is the volatile component of the aromatherapy composition.

Persons of ordinary skill in the art will readily appreciate that there are several ways for performing the contacting step. One illustrative non-limiting example would be to contact the particulate component with the essential oil composition under conditions, and for period of time, wherein the particulate component retains an amount of the essential oil composition. The retention is manifested after the particulate component and its retained amount of essential oil is separated from that portion of the essential oil composition that did not associate sufficiently to be retained.

An additional illustrative but non-limiting manner of performing the contacting step is to spray an amount of the particulate component with at least one essential oil. The spraying could be performed by varying known parameters that may affect the amount of the one or more specific essential oils actually retained by a specific type of particulate component. Additional methods of soaking, spraying, coating, brushing of particles with essential oils are known in the art.

### DEFINITIONS

The “particulate component” of the device comprises one or more particles having either a regular or irregular shape. The term particle refers to an individual grain or granule-like structure. The particulate component may comprise one or more individual particles that are solid, porous, absorbent, semisolid or gel-like and having a substantially rounded shape. The term particle as used herein also encompasses irregularly shaped bits of matter, e.g., star-shaped, pebble-shaped, etc. Further, the particulate component or particles have the property of retaining amounts of essential oils. The retained amount is that amount of at least one essential oil that associates with the particle after, (a) the particles have been contacted with compositions comprising at least one essential oil, and (b) the essential oil-contacted particles are subsequently separated from the remainder of the essential oil comprising composition. The term “particle” does not intend to connote or infer any particular size range of particles used in the inventive subject matter. For example, particles may have an average diameter of approximately 0.01 mm to about 25 mm. However, persons of ordinary skill in the art would appreciate that particles suitable for use in the invention may encompass various sizes and shapes.

Essential oils comprise a class of oils that typically impart the characteristic odor of a plant from which the oil is obtained. Accordingly, the term “volatile component” refers to the volatile and/or aromatic substances present in an aromatherapy composition that are inhaled or released to the ambient atmosphere.

An essential oil may be used in perfumes or fragrance mixtures, food flavorings and aromatherapy and may also be referred to herein as a “volatile oil” or, an “aromatic oil.” The volatile oil is derived from odorous plant materials. Essential oils generally constitute the aromatic components of the plants in which they exist. Essential oils are either distilled (water, steam or dry) or expressed by pressure from the plant material used as the source of the essential oil.

The table below provides a non-limiting list of essential oils. It would be clearly appreciated by persons of ordinary skill in the art that this list will very likely increase as new oils are prepared.

TABLE I
Non-Limiting List of Essential Oils
and Corresponding Plant Sources*

| | |
|---|---|
| Basil | (Ocimum basilicum) |
| Bergamot | (Citrus bergamia) |
| Black Spruce | (Picea mariana) |
| Cedarwood | (Cedrus atlantica) |
| Chamomile | (Anthemis nobilis) |
| Champaca | (Michelea champaca) |
| Clary Sage | (Salvia sclarea) |
| Coriander | (Coriandrum sativum) |
| Fir Needle | (Abies alba) |
| Frankincense | (Boswellia carterii) |
| Frangipani | (Plumeria rubra) |
| Geranium | (Pelargonium graveoloens) |
| Ginger | (Zingiber officinale) |
| Grapefruit | (Citrus paradisi) |
| Immortelle | (Helichrysum angustifolium) |
| Jasmine | (Jasminum officinalis) |
| Juniper | (Juniperus communis |
| Lavender | (Lavandula augustifolia) |
| Mandarin | (Citrus madurensis) |
| Marjoram | (Origanum majorana) |
| Mimosa | (Acacia dealbata) |
| Myrrh | (Commiphora myrrha) |
| Neroli | (Citrus aurantium) |
| Patchouli | (Pogostemon cablin) |
| Peppermint | (Mentha piperita) |
| Petitgrain | (Citrus aurantium) |
| Pine Needle | (Pinus sylvestris) |
| Rose | (Rosa centifolia) |
| Rosemary | (Rosmarinus officinalis) |
| Rosewood | (Aniba roseodora) |
| Sandalwood | (Santalum album) |
| Spikenard | (Nardastachus jatamansi) |
| Vanilla | (Vanilla planifolia) |
| Vetivert | (Vetiveria zizanoides |

*Plant sources of essential oil are in parentheses.

Further, the particles may be referred to as “spun wood particles,” “spun wood granules” or “spun wood beads” and the like. As described below, the term “spun” reflects that the particles may be prepared, in part, by a variety of methods wherein wood pulp is converted to modified or unmodified cellulose fibers by solvent spinning.

In another embodiment, the particles may be a bead or rounded piece fashioned from, e.g., wood that has been processed by conventional lathing techniques.

The particles may be contacted with the essential oils in different ways, e.g., bathing or washing the particles in a composition comprising at least one essential oil. Alternatively, the particles may be sprayed with an essential oil composition. Persons of ordinary skill in the art would appreciate that essential oils may be contacted with various particles under conditions suitable for obtaining the desired amount of retained essential oils.

The amount of retained essential oils is that which remains associated with the particles after contacting the particles with one or more essential oils, and removal of the essential oil not associated with particles. Persons of ordinary skill in the art would appreciate that particles made of different materials different physical configuration and dimensions would have different essential oil-retaining properties, and may be selected according to specific applications or preferences.

An “effective amount” is that amount of inhaled essential oil required to affect one or more of the benefits of aromatherapy. The effects of, or responses to aromatherapy with essential oils are known to include, but are not limited to, psychological and physical perception of well-being, enhanced relaxation, increased calm or tranquility, decrease in anxiety and/or depression, and the alleviation of pain.

The term “container” refers to any device or vessel that is suitable for holding the aromatherapy composition. The container may be glass, a metal or metal alloy, plastic, ceramic or wood or any combination of thereof.

The term “subject” refers to an individual that desires or needs the benefits of aromatherapy. Most often, a subject will be a human being. However persons of ordinary skill in the art will appreciate that the term “subject” also applies to various animals, most commonly those used as domestic pets.

The aromatherapy administering device functions, in part, as an inhalation device.

The device comprises a container or housing structure to hold the aromatherapy composition. The container comprises one or more apertures, holes, slits or other openings, through which the volatile component of the aromatherapy composition escapes from the container. The escaped volatile component of the aromatherapy composition would likely enter either the ambient atmosphere or into a subject that inhales or breathes the escaped volatile component directly through the subject's nose or mouth that is positioned in proximity to the one or more apertures, holes, slits or other openings. The container also comprises a cap, an orifice reducer or other conventional sealing mechanism to cover the one or more apertures, holes, slits or other openings.

In one embodiment the inventive subject matter discloses a device suitable for administering aromatherapy to a subject, the device comprising,
(a) an aromatherapy composition comprising at least one particle that has been contacted with at least one essential oil, wherein the particle retains an amount thereof; and
(b) a container for housing the aromatherapy composition, the container having at least one opening to allow the escape of the aromatherapy's volatile component from the container.

Another embodiment of the device described and claimed herein is an inhaler suitable for use in aromatherapy comprising;
(a) an aromatherapy composition comprising a plurality of particles that have been contacted with at least one essential oil, wherein the particles retain an amount thereof; and
(b) a cap or other sealing mechanism covering one or more openings or apertures in the container, thereby preventing undesired release of volatile components to the atmosphere.

The particles may be of any shape including irregularly shaped, or being of a completely non-uniform or asymmetrical shape. However, depending in the mode of manufacture and the starting material, a substantially spherical or elliptical contour may be most convenient to prepare.

The particles may have a substantially smooth surface. Alternatively, the particles may also be modified to enhance the retention of essential oils. For example, the surface may be grooved or ridged along the outermost surface of the particle. Alternatively, the particles may comprise small conduits or tunnels that extend from the outer surface of the particle toward the interior or center of the particles, thereby creating spaces to form reservoirs of essential oils within the bead. Alternatively, the particle may be prepared from absorbent materials that upon contact with the essential oils, allow the essential oils to permeate throughout the particle.

Persons of ordinary skill in the art would readily appreciate that one or more of the aforementioned strategies for enhancing the absorptive properties of the particles toward essential oils may be implemented in preparing a desired type of particle.

The particles for the disclosed aromatherapy inhaler may be prepared from natural materials or synthetic polymers or plastics. In fact, the particles may also be made of a mixture of natural and synthetic components. In other embodiments, the particulate bead-like particles may be prepared using a mixture of natural and synthetic materials. Persons of ordinary skill in the art would recognize that suitable particles may be made of wood, plastics, ceramics, clays and similar porous materials or other ceramics.

Regardless of the material used, the aromatic oil may become associated with the particle by means of dipping, spraying or brushing.

Another embodiment of the device described and claimed herein is an inhaler suitable for use in aromatherapy comprising;
(a) an aromatherapy composition comprising a plurality of particles that have been contacted with at least one essential oil, wherein the particles retain an amount thereof;
(b) wherein the particles comprise natural and synthetic materials such as wood, plastics, ceramics, clays and other known porous materials; and
a cap or other sealing mechanism covering one or more openings or apertures in the container, thereby preventing undesired release of volatile components to the atmosphere.

A non-limiting illustrative example of a suitable particle is a bead-like structure comprising solvent spun cellulose-containing fibers. This material is available from Orlandi Inc., of (Farmingdale, N.Y.). There are likely to exists various modifications of the methods to prepare such materials, and persons of ordinary skill in the art would likely be able to develop further modification of the methods to prepare the material without substantially altering the nature of the resultant fiber.

A broad overview of the manufacturing process encompasses preparing wood pulp as a starting material. The pulp may then be dried and formed into sheets. The dried material is treated with a solvent, e.g., an amine oxide until the cellulose forms a clear solution that may be filtered. The solution is pumped through the dies of a spinneret to form spun fibers. It is the passing of the cellulose through the dies that is referred to as “spinning” and is a well-known procedure in the general manufacture of nonwoven fabrics. (U.S. Pat. No. 5,725,821 to Gannon, et al.) The fibers are dried and may then be used in many ways, e.g., as fibers, or in a granulated or ground form prepared from the fibers. Further, the powder or granules may be compressed and/or shaped in particles of varying size and shapes.

The wooden beads allow for ideal retention and volatility of the blends. They will last for about 6 months.

Thus, an embodiment of the device suitable for administering aromatherapy to a subject, comprises an aromatherapy composition comprising,
(a) at least one particle comprising spun wood, wherein the particle has been contacted with a composition comprising at least one essential oil; and
(b) a volatile component comprising the amount of the at least one essential oil retained by the particle,
wherein the aromatherapy composition is provided in a container, the container having at least one opening to allow the escape of a portion of the volatile component.

The user will benefit from volatile essential oil molecules without the inconvenience of spillage or direct contact with skin. In addition, the preferred materials for preparing the essential oil-retaining particles will not to contain chemicals that are unsuitable or even hazardous for repeated deep breathing.

The following descriptions provide only illustrative examples of the combination of an essential oil and a particle for aromatherapy. As stated above, many materials may provide a useful substrate for retaining essential oils. The following illustrations are not meant to limit the claimed combination to these specific examples.

In one embodiment, the invention encompasses an aromatherapy combination, comprising (1) at least one essential oil, and (2) a particle that is permeated, impregnated or otherwise physically retains an amount of the at least one essential oil.

In an additional embodiment, the invention encompasses an aromatherapy combination, comprising (1) at least one essential oil, and (2) a cellulose-containing particle or a chemically-modified cellulose-containing particle, which is contacted or permeated or impregnated with an amount of the at least one essential oil, so that the particle may release the volatile component of the at least one essential oil at the desired time.

It is noted that the foregoing descriptions and illustrations are provided for illustrative purposes only. The disclosure is not meant to limit the scope of the claimed subject matter in any way. Persons of ordinary skill in the art will readily appreciate that based on the guidance provided herein, a multitude of obvious or trivial modifications of the claimed subject matter may be prepared, and yet remain within the scope of the properly construed claims.

What is claimed is:

1. An aromatherapy composition comprising,
(a) at least one essential oil, and
(b) a particle suitable for retaining an effective amount of the at least one essential oil,
wherein, the composition is formed by contacting the particle with the at least one essential oil.

2. The composition of claim 1, wherein the particle comprises at least one material selected from the group consisting of wood, clay, plastic, silicone, ceramic and glass.

3. The composition of claim 1, wherein the particle comprises a wooden bead.

4. The composition of claim 1, wherein the particle comprises a spun wood particle or spun wood bead.

5. The composition of claim 1, wherein the particle is made of material that permits the essential oil to permeate the particle.

6. The composition of claim 1, wherein the at least one essential oil is selected from the group consisting of,

| | |
|---|---|
| Basil | (Ocimum basilicum), |
| Bergamot | (Citrus bergamia), |
| Black Spruce | (Picea mariana), |
| Cedarwood | (Cedrus atlantica), |
| Chamomile | (Anthemis nobilis), |
| Champaca | (Michelea champaca), |
| Clary Sage | (Salvia sclarea), |
| Coriander | (Coriandrum sativum), |
| Fir Needle | (Abies alba), |
| Frankincense | (Boswellia carterii), |
| Frangipani | (Plumeria rubra), |
| Geranium | (Pelargonium graveoloens), |
| Ginger | (Zingiber officinale), |
| Grapefruit | (Citrus paradisi), |
| Immortelle | (Helichrysum angustifolium), |
| Jasmine | (Jasminum officinalis), |
| Juniper | (Juniperus communis), |
| Lavender | (Lavandula augustifolia), |
| Mandarin | (Citrus madurensis), |
| Marjoram | (Origanum majorana), |
| Mimosa | (Acacia dealbata), |
| Myrrh | (Commiphora myrrha), |
| Neroli | (Citrus aurantium), |
| Patchouli | (Pogostemon cablin), |
| Peppermint | (Mentha piperita), |
| Petitgrain | (Citrus aurantium), |
| Pine Needle | (Pinus sylvestris), |
| Rose | (Rosa centifolia), |
| Rosemary | (Rosmarinus officinalis), |
| Rosewood | (Aniba roseodora), |
| Sandalwood | (Santalum album), |
| Spikenard | (Nardastachus jatamansi), |
| Vanilla | (Vanilla planifolia) and |
| Vetivert | (Vetiveria zizanoides). |

7. The composition of claim 1, comprising a vessel or container to house the composition, the vessel or container further comprising a sealing mechanism.

8. The composition of claim 7, wherein the sealing mechanism comprises a cap.

9. A device suitable for administering aromatherapy to a subject, the device comprising, an aromatherapy composition comprising
(a) a particulate component that has been contacted with at least one essential oil under conditions wherein the particulate component retains an amount of the at least one essential oil; and
(b) a volatile component comprising the retained amount of the at least one essential oil,
wherein, the aromatherapy composition is provided in a container, the container having at least one opening to allow the escape of a portion of the volatile component.

10. The device of claim 9 wherein the at least one particle is a beadlike piece comprising at least one material selected from the group consisting of wood, clay, plastic, silicone, ceramic and glass.

11. The device of claim 9 wherein the at least one particle is a beadlike piece comprising spun wood.

12. The device of claim 9 wherein the at least one essential oil is selected from the group consisting of,

| | |
|---|---|
| Basil | (Ocimum basilicum), |
| Bergamot | (Citrus bergamia), |
| Black Spruce | (Picea mariana), |
| Cedarwood | (Cedrus atlantica), |
| Chamomile | (Anthemis nobilis), |
| Champaca | (Michelea champaca), |
| Clary Sage | (Salvia sclarea), |
| Coriander | (Coriandrum sativum), |
| Fir Needle | (Abies alba), |
| Frankincense | (Boswellia carterii), |
| Frangipani | (Plumeria rubra), |
| Geranium | (Pelargonium graveoloens), |
| Ginger | (Zingiber officinale), |
| Grapefruit | (Citrus paradisi), |
| Immortelle | (Helichrysum angustifolium), |
| Jasmine | (Jasminum officinalis), |
| Juniper | (Juniperus communis), |
| Lavender | (Lavandula augustifolia), |
| Mandarin | (Citrus madurensis), |
| Marjoram | (Origanum majorana), |
| Mimosa | (Acacia dealbata), |
| Myrrh | (Commiphora myrrha), |
| Neroli | (Citrus aurantium), |
| Patchouli | (Pogostemon cablin), |
| Peppermint | (Mentha piperita), |
| Petitgrain | (Citrus aurantium), |
| Pine Needle | (Pinus sylvestris), |
| Rose | (Rosa centifolia), |
| Rosemary | (Rosmarinus officinalis), |
| Rosewood | (Aniba roseodora), |
| Sandalwood | (Santalum album), |
| Spikenard | (Nardastachus jatamansi), |
| Vanilla | (Vanilla planifolia) and |
| Vetivert | (Vetiveria zizanoides). |

13. The administering of aromatherapy to a subject using the device of claim 9, the method comprising,
providing the device of claim 9 to a subject, and
inhaling by the subject of an amount of the volatile component of the aromatherapy composition within the device of claim 9.

14. A device suitable for administering aromatherapy to a subject, the device comprising, an aromatherapy composition comprising
(c) at least one particle comprising spun wood, wherein the particle has been contacted with a composition comprising at least one essential oil; and
(d) a volatile component comprising the amount of the at least one essential oil retained by the particle,
wherein the aromatherapy composition is provided in a container, the container having at least one opening to allow the escape of a portion of the volatile component.

15. The device of claim 14, wherein the at least one essential oil is selected from the group consisting of,

| | |
|---|---|
| Basil | (Ocimum basilicum), |
| Bergamot | (Citrus bergamia), |
| Black Spruce | (Picea mariana), |
| Cedarwood | (Cedrus atlantica), |
| Chamomile | (Anthemis nobilis), |
| Champaca | (Michelea champaca), |
| Clary Sage | (Salvia sclarea), |
| Coriander | (Coriandrum sativum), |
| Fir Needle | (Abies alba), |
| Frankincense | (Boswellia carterii), |
| Frangipani | (Plumeria rubra), |
| Geranium | (Pelargonium graveoloens), |
| Ginger | (Zingiber officinale), |
| Grapefruit | (Citrus paradisi), |
| Immortelle | (Helichrysum angustifolium), |
| Jasmine | (Jasminum officinalis), |
| Juniper | (Juniperus communis), |
| Lavender | (Lavandula augustifolia), |
| Mandarin | (Citrus madurensis), |
| Marjoram | (Origanum majorana), |
| Mimosa | (Acacia dealbata), |
| Myrrh | (Commiphora myrrha), |
| Neroli | (Citrus aurantium), |
| Patchouli | (Pogostemon cablin), |
| Peppermint | (Mentha piperita), |
| Petitgrain | (Citrus aurantium), |
| Pine Needle | (Pinus sylvestris), |
| Rose | (Rosa centifolia), |
| Rosemary | (Rosmarinus officinalis), |
| Rosewood | (Aniba roseodora), |
| Sandalwood | (Santalum album), |
| Spikenard | (Nardastachus jatamansi), |
| Vanilla | (Vanilla planifolia) and |
| Vetivert | (Vetiveria zizanoides). |

16. The device of claim 14 wherein the container is made of glass, a metal or metal alloy, plastic, ceramic or wood.

17. The administering of aromatherapy to a subject using the device of claim 14, the method comprising,
the subject inhaling of an amount of the volatile component of the aromatherapy composition from the device of claim 14.

18. A method of administering aromatherapy to a subject comprising the steps of,
(a) providing a subject a device suitable for administering aromatherapy, the device comprising, an aromatherapy composition comprising, at least one particle, wherein the particle has been contacted with a composition comprising at least one essential oil; and a volatile component comprising the amount of the at least one essential oil retained by the particle, wherein the aromatherapy composition is provided in a container having at least one opening to allow the escape of a portion of the volatile component, and
(b) inhaling or breathing an amount of the volatile component through the at least one opening.

19. The method of claim 18 wherein the particle comprises spun wood.

20. The method of claim 19, wherein the at least one essential oil is selected from the group consisting of,

| | |
|---|---|
| Basil | (Ocimum basilicum), |
| Bergamot | (Citrus bergamia), |
| Black Spruce | (Picea mariana), |
| Cedarwood | (Cedrus atlantica), |
| Chamomile | (Anthemis nobilis), |
| Champaca | (Michelea champaca), |
| Clary Sage | (Salvia sclarea), |
| Coriander | (Coriandrum sativum), |
| Fir Needle | (Abies alba), |
| Frankincense | (Boswellia carterii), |
| Frangipani | (Plumeria rubra), |
| Geranium | (Pelargonium graveoloens), |
| Ginger | (Zingiber officinale), |
| Grapefruit | (Citrus paradisi), |
| Immortelle | (Helichrysum angustifolium), |
| Jasmine | (Jasminum officinalis), |
| Juniper | (Juniperus communis), |
| Lavender | (Lavandula augustifolia), |
| Mandarin | (Citrus madurensis), |
| Marjoram | (Origanum majorana), |
| Mimosa | (Acacia dealbata), |
| Myrrh | (Commiphora myrrha), |
| Neroli | (Citrus aurantium), |
| Patchouli | (Pogostemon cablin), |
| Peppermint | (Mentha piperita), |
| Petitgrain | (Citrus aurantium), |
| Pine Needle | (Pinus sylvestris), |
| Rose | (Rosa centifolia), |
| Rosemary | (Rosmarinus officinalis), |
| Rosewood | (Aniba roseodora), |
| Sandal wood | (Santalum album), |
| Spikenard | (Nardastachus jatamansi), |
| Vanilla | (Vanilla planifolia) and |
| Vetivert | (Vetiveria zizanoides). |
